(19) United States
(12) Patent Application Publication
(10) Pub. No.: US 2003/0021805 A1
(43) Pub. Date: Jan. 30, 2003

(54) Generation of HCV-like particles and chimeric HCV virus

(76) Inventor: Glen N. Barber, Miami, FL (US)

Correspondence Address: VENABLE, BAETJER, HOWARD AND CIVILETTI, LLP, P.O. BOX 34385, WASHINGTON, DC, 20043-9998, US

(21) Appl. No.: 10/156,647

(22) Filed: May 29, 2002

Related U.S. Application Data
(60) Provisional application No. 60/293,532, filed on May 29, 2001.

(51) Int. Cl. C12P 21/06; A61K 39/12; A61K 39/29; C12N 15/00; C12N 15/09; C12N 15/63; C12N 15/70; C12N 15/74

(52) U.S. Cl. 424/199.1; 424/204.1; 424/225.1; 435/320.1; 435/69.1

(57) ABSTRACT

A recombinant vesicular stomatitis virus (VSV) that expresses Hepatitis C virus structural proteins, an immunogenic composition and vaccine containing the recombinant VSV, and a method of preventing or treating HCV.

This application claims priority to U.S. provisional application No. 60/293,532, filed May 29, 2001, which is hereby incorporated by reference.

### BACKGROUND OF THE INVENTION

1. Field of the Invention

The invention relates to a recombinant vesicular stomatitis virus (VSV) that expresses Hepatitis C virus structural proteins, an immunogenic composition and vaccine containing the recombinant VSV, and a method of preventing or treating Hepatitis C virus.

2. Background Information

Hepatitis C virus (HCV), a positive stranded RNA virus of the flaviviridae family, is estimated to infect at least 400 million people worldwide and is a major etiologic agent of hepatocellular carcinoma (HCC) and liver failure (9). Standard therapeutic intervention consists of the administration of interferon in combination with ribavarin. However, less than 50% of infected patients respond to this regimen and few alternative therapies exist. There is presently no tissue culture system to efficiently cultivate HCV, which not only hampers research efforts aimed at elucidating the molecular mechanisms of virus replication but also impedes attempts at producing candidate vaccines and immunotherapies that target HCV-related disease. Consequently, a number of recombinant sub-unit based HCV vaccine strategies, involving genetic immunization and purified proteins, have been attempted (2, 3, 7, 8, 12, 17). Determining the ideal vaccination strategy has been made more difficult since the type of immune response considered effective for the eradication of HCV infection, including natural eradication of HCV or viral clearance resulting from interferon (IFN) therapy, presently remains to be fully determined. Furthermore, the heterogeneity between multiple HCV genotypes and the generation of quasispecies indicate that cross-protection between HCV strains may be problematic (6).

Nevertheless, studies have indicated that recombinant HCV envelope glycoproteins E1 and E2 are able to elicit protective immunity against homologous virus challenge in chimpanzees, an effect thought to be mediated by the generation of anti-E2 antibodies. Significant evidence also indicates that early, vigorous and sustained Th1 and multispecific cytotoxic T-cell (CTL) responses are further critical for the elimination of HCV infection (4, 5). Collectively, the data would therefore indicate that an optimum HCV vaccine or post-therapeutic strategy should not only induce a potent humoral response to neutralize virus infection but should also elicit a strong, broad range CTL response to limit virus amplification and spread.

Recently, a procedure for generating replication-competent, negative-stranded vesicular stomatitis virus entirely from cDNA has been established (11). U.S. Pat. No. 6,168,943 discloses a method for making recombinant VSV that expresses foreign proteins. The genetic malleability of VSV has allowed the development of recombinant VSVs that express foreign viral proteins to high levels (10, 16).

The generation of recombinant VSV has been evaluated in a number of vaccine strategies designed to prevent virus infection. For example, live attenuated VSV expressing the human immunodeficiency virus (HIV) envelope (env) and core (gag) proteins has been shown to protect rhesus monkeys from acquired immunodeficiency syndrome (AIDS) following challenge with pathogenic SHIV (14). Similarly, VSV expressing influenza virus or measles hemagglutinin protein conferred resistance to lethal influenza virus or measles virus infection, respectively (13, 15).

One of the advantages of using a recombinant VSV system for vaccine studies is that the virus is relatively innocuous and naturally occurring human infections are rare. The apparent seroprevalence of VSV antibodies are generally low within the human population. Furthermore, the genetic malleability of VSV indicates that large, multiple inserts of foreign genes can be achieved that are expressed to high levels, without dramatically affecting virus growth. In preliminary vaccine studies, VSV has been found to elicit strong humoral and cellular immune responses. VSV has a simple genetic constitution of only 5 genes and is unable to undergo reassortment or integration. These events confer additional advantages of VSV over other live virus vaccine systems presently in use.

### SUMMARY OF THE INVENTION

It is one object of the invention to provide a recombinant vesicular stomatitis virus (VSV) that expresses HCV structural proteins. In a preferred embodiment, the recombinant VSV expresses one or more of the Core, E1 and E2 proteins of HCV (NIHJ1). In a particularly preferred embodiment, the recombinant VSV expresses the contiguous Core, E1 and E2 polyprotein of HCV. This polyprotein may then be fully processed into the individual HCV structural proteins.

In one embodiment, this object is accomplished by inserting the contiguous Core, E1 and E2 coding region of HCV (NIHJ1) into the VSV genome. The recombinant VSV of the invention (designated as VSV-HCV-C/E1/E2) grows to high titers in vitro and efficiently expresses the incorporated HCV polyprotein, which becomes fully processed into the individual HCV structural proteins.

Accordingly, it is a further object of the invention to provide a recombinant VSV comprising the contiguous Core, E1 and E2 coding region of HCV (NIHJ1). Preferably the recombinant VSV is capable of replication and expressing HCV structural proteins when introduced into a mammal.

As indicated by biochemical and biophysical analysis, the HCV Core, E1 and E2 proteins of the recombinant VSV reassemble to form virus-like particles similar to the ultrastructural properties of HCV virions. Accordingly, it is another object of the invention to provide a recombinant VSV that produces virus-like particles with properties of HCV virions (HCV-LPs). In addition to ultrastructural properties, such properties include, for example, the ability to elicit a cell-mediated and/or humoral immune response when administered to a mammal, preferably a human.

Mice immunized with VSV-HCV-C/E1/E2 generate cell-mediated immune responses to all of the HCV structural proteins and humoral responses to Core and E2. Mammalian cell-generated VSV expressing HCV Core, E1, and E2 and HCV-LPs is antigenic and immunogenic, and is therefore expected to be a key component in vaccine strategies designed to prevent HCV infection.

Thus, it is a further object of the invention to provide a vaccine or an immunogenic composition capable of eliciting and immune response to HCV when administered to an individual in an effective dosage. The vaccine or immunogenic composition of the invention is expected to be useful for both treatment and prophylaxis of HCV infection. The vaccine or immunogenic composition of the invention may comprise the recombinant VSV and/or the HCV virus like particles of the invention. In one preferred embodiment, the vaccine or immunogenic composition comprises VSV-HCV-C/E1/E2. In another preferred embodiment the vaccine or immunogenic composition comprises HCV-LPs produced by VSV-HCV-C/E1/E2. The vaccine or immunogenic composition of the invention may also include physiologically acceptable excipients, adjuvants and carriers, as will be familiar to those of skill in the art.

The vaccine or immunogenic composition will be administered in a manner compatible with the dosage formulation, and in an amount that is therapeutically effective and immunogenic. The quantity administered will depend on the individual to be treated, including, for example, the capacity of the individual's immune system to respond. The precise amounts of active ingredient administered can be determined by a skilled practitioner without undue experimentation.

The vaccine or immunogenic composition of the present invention may also include one or more adjuvants, excipients and carriers as is customary in the art. Possible adjuvants include Freund's adjuvant (complete or incomplete), aluminum compounds, such as aluminum hydroxide, aluminum phosphate, aluminum monostearate and interferon. Possible carriers include saline, buffered saline, dextrose, water, glycerol, sterile isotonic aqueous buffer, and combinations thereof. A more detailed description and additional examples of suitable vaccine components and formulations can be found in Remington's Pharmaceutical Sciences, Mack Publishing Company, Easton, Pa., 18th Edition, 1990, a standard reference in this field.

It is another object of the invention to provide a method of inducing an immune response to HCV in an individual, said method comprising administering to the individual an effective amount of the recombinant VSV of the invention, optionally with additional adjuvants, excipients and carriers, as detailed hereinabove. In a preferred embodiment, VSV-HCV-C/E1/E2 is administered.

It is another object of the invention to provide a method of inducing an immune response to HCV in an individual, said method comprising administering to the individual an effective amount of the HCV virus-like particles of the invention. In a preferred embodiment, HCV LPs produced by VSV-HCV-C/E1/E2 are administered.

The vaccine or immunogenic composition of the invention may be administered by any suitable route, including, for example, orally, by intramuscular or intradermal injection, suppository, by inhalation, to nasal mucosa, or lungs. Persons of skill in the art will be familiar with suitable routes of administration, and with suitable formulations. The vaccine or immunogenic composition may also be administered ex vivo on dendritic cells. Effective and safe dosages can be determined by the skilled practitioner using routine experimentation.

In a broad aspect, the invention provides a recombinant vesicular stomatitis virus that expresses structural or nonstructural proteins of Hepatitis C virus. For example, using the methods described herein, recombinant VSV can be made that expresses one or more of non-structural proteins NS2, NS3, NS4a, NS4b, NS5a and NS5b, optionally in combination with structural proteins such as Core, E1 and E2. Such a virus might express structural proteins with some or all of the non-structural proteins, and would induce cell-mediated and humoral activity to the structural and non-structural proteins.

Both recombinant VSV and VLPs can be made representative of any strain of HCV. Such strains are familiar to those of skill in the art. By the use of techniques described hereinbelow and familiar to those of skill in the art, VLPs may also be composed of HCV structural proteins of different strains. Also included in the invention are replication-defective VSV expressing HCV proteins or immunomodulatory genes to enhance an immune response. VSV-HCV or VLPs can be used to transfect or target antigen-presenting cells. VSV can also be generated to make replication competent HCV (replicon system).

Recombinant VSV may also be generated to express any strain of HCV, or to contain one or more of the structural proteins from one or more strains of HCV. For example, VSV containing the Core, E1 sand E2 from strain 1b and 1a (e.g. two sets of one or more structural proteins within the same virus) can be generated. Such a virus would confer cell-mediated and humoral activity to a variety of strains of HCV. This is important as presently quasispecies varients of HVC present a problem in immunization. A vaccine to strain A may not confer protection to strain B. This problem could be overcome with the development of multistrain VSV-HCV combinations. Furthermore, any VLPs produced by VSV expressing C/E1/E2 from strain 1a as well as strain 1b would be VLP chimeras themselves, composed of C/E1/E2 representing two strains. This would broaden the immune response, potentially eliciting immunity to a number of HCV strains.

VSV can also be genetically engineered to express the HCV envelope or other proteins on its surface. For example, it is possible to switch the VSV envelope G for HCV E1/E2 (or include E1 and E2 on the surface of VSV by attaching the G transmembrane region to the c-terminus of E1/E2) to create a VSV/HCV hybrid that can be used for vaccine and immunotherapeutic purposes.

In examples described hereinbelow, HCV VLPs are expressed from VSV that also expresses VSV G. The VLPs so expressed may be comprised of Core/E1/E2 with some VSV G, which may be advantageous for certain applications because VSV G is extremely tropic for a number of tissue types, while HCV E1/E2 is not. Thus, VLPs containing VSV G may be additionally immunogenic and be taken up by antigen presenting cells.

VSV lacking VSV G (VSVΔG/HCV Core/E1/E2) is also included in the invention. This construct can be made in helper cells expressing VSV G to create a virus that has G on the surface. Following infection of target cell, in vitro or in vivo, VSV infects and replicates HCV proteins and its own (but no G). Such viruses are replication-defective and cannot infect a second set of cells because they have no receptor attachment protein (VSV G). They may be safer and more acceptable for vaccine formulations. Secondly, VLPs made from such a ΔG virus would have no G protein in their composition and be pure Core/E1/E1 particles. Other replication defective VSVs that express HCV proteins are also included in the invention, for example, VSV expressing HCV proteins (any or combinations) that are mutated or lack any of the VSV N, P, M, G, and L proteins.

### BRIEF DESCRIPTION OF THE DRAWINGS

FIG. 1A. Construction of rVSV expressing HCV Core, E1, and E2.

FIG. 1B. Growth analysis of VSV-XN2, VSV-GFP, and VSV-C/E1/E2.

FIG. 1C. Expression of HCV Core, E1, and E2 by VSV-C/E1/E2 in BHK cells. BHK cells were infected at an m.o.i. of 1 for 18 hrs with either VSV-XN2 or VSV-C/E1/E2. Infected cells were then lysed and protein expression determined by immunoblot analysis as previously described.

FIG. 1D. HCV structural proteins are localized to the perinuclear region of VSV-C/E1/E2 infected liver cells.

FIGS. 2A-B. HCV E2 is not associated with VSV-HCV-C/E1/E2. Cell medium from VSV-HCV-C/E1/E2 or control virus infected cells (concentrated by ultracentrifugation) was immunoprecipitated with a sheep antibody to VSV G (Biogenesis) or a goat antibody to HCV E2 (Immunodiagnostics Inc.). (A) After SDS-PAGE, immunoblot against mouse antiserum raised to VSV. (B) Reprobe against mouse antiserum to HCV E2, demonstrating absence of E2 in VSV complexes.

FIG. 2C. Gradient purified HCV Core, E1 and E2 form complexes. Sucrose gradient fractions containing HCV-LPs were identified by immunoblot and immunoprecipitated using E2 specific antibody. Complexes were washed, analyzed by SDS PAGE and immunoblotted using antibody to HCV Core, E1 and E2.

FIG. 2D. Co-immunoprecipitation analysis of VSV-HCV-C-E1/E2 infected cells. 35S-methioine/cysteine laveled lysates (600 μCi/4 hours from mock, VSV-XN2, or VSV-C/E1/E2 were immunoprecipitated with mouse antiserum raised to VSV or anti-E2 mAb, or normal mouse IgG. Complexes were washed, analyzed by SDS-PAGE and visualized by autoradiography.

FIG. 2E. Sucrose gradient demonstrating the co-sedimentation of the HCV structural proteins Core, E1, and E2 in fractions 14-20.

FIG. 3A. Electron microscopy images of BHK cells infected with VSV-C/E1/E2 demonstrate the formation of HCV-LPs in cytoplasmic vacuoles. n denotes nucleus. Black arrows indicate HCV-LPs in vacuoles formed from rough endoplasmic reticulum. * indicates enlarged inset. Bar in inset is 100 nm. White arrow indicates immature VSV particles.

FIG. 3B. Electron microscopy images of HCV-LPs purified by equilibrium sedimentation sucrose gradient as in FIG. 2E. Bar inset is 50 nm.

FIGS. 4A-4D. VSV expressing HCV structural protein generates both a humoral and cellular response. 4A. Core specific ELISA demonstrating the generation of antibodies to the Core protein by VSV-C/E1/E2 vaccinated mice. Each bar represents a pool of three mice. 4B. E2 specific ELISA demonstrating a strong antibody response to E2 by VSV-C/E1/E2 vaccinated mice but not VSV-GFP or PBS injected mice. Each bar represents a pool of three mice. 4C. CD4⁺T cells from VSV-C/E1/E2 vaccinated mice become activated and proliferate in response to purified Core protein in vitro. 4D. Mice vaccinated with VSV-C/E1/E2 generate CTLs against epitopes in the Core, E1, and E2 proteins. ELISPOT analysis indicates that CTLs become activated and secrete IFN-γ in response to CTL specific HCV peptides.

### DETAILED DESCRIPTION OF THE INVENTION

The embodiments illustrated and discussed in the present specification are intended only to teach those skilled in the art the best way known to the inventors to make and use the invention, and should not be considered as limiting the scope of the present invention. The exemplified embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

### EXAMPLE 1

Generation of rVSV Expressing HCV Core, E1 and E2.

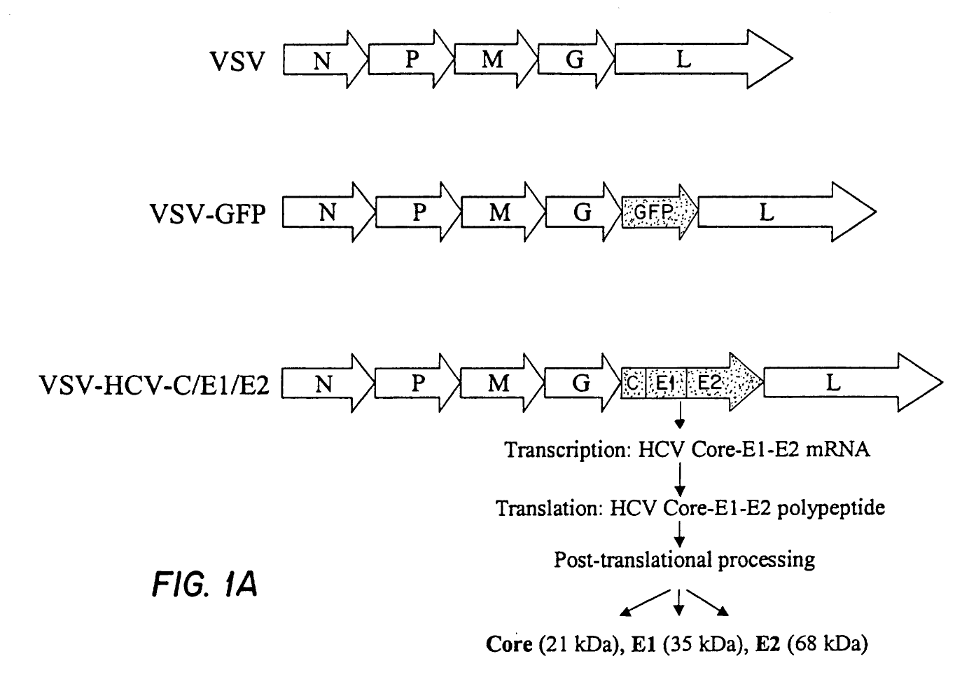

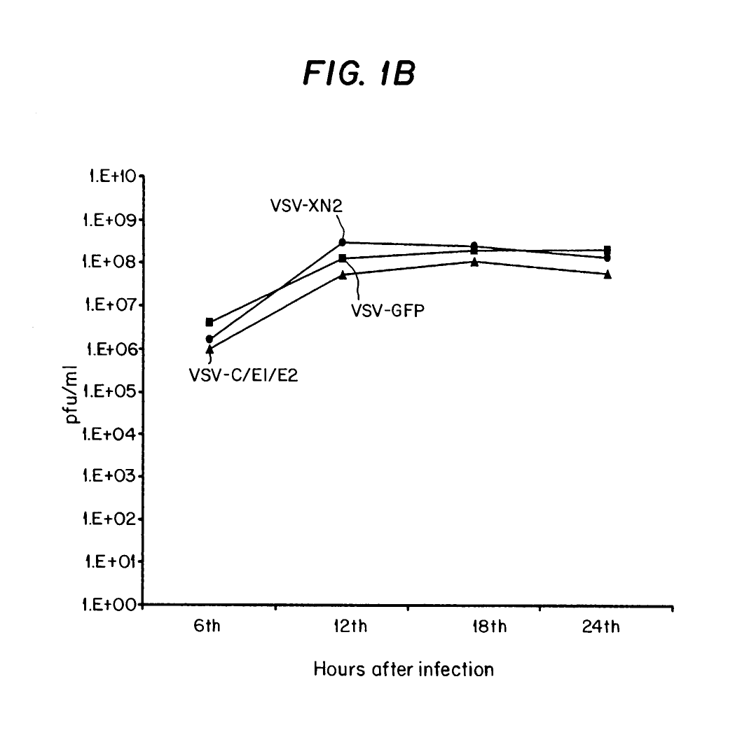

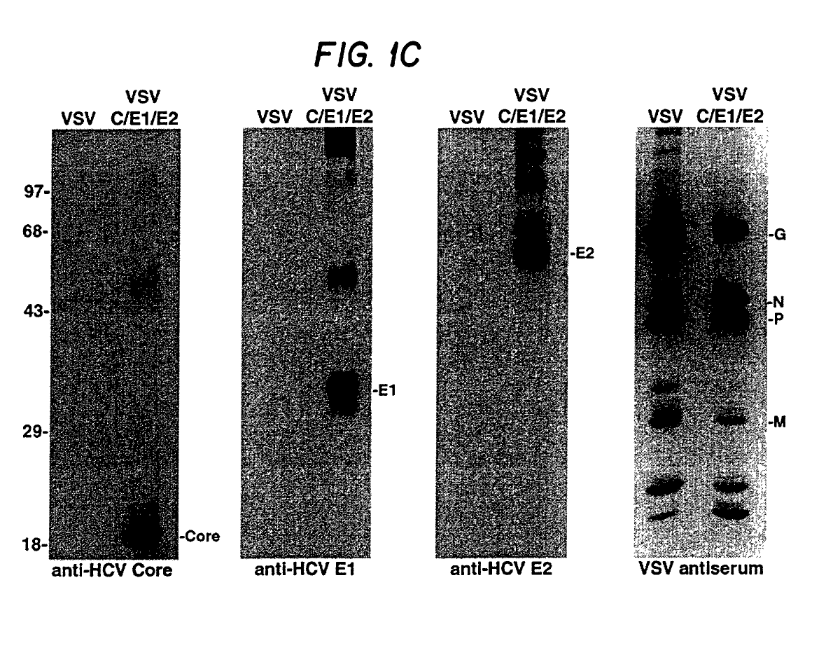

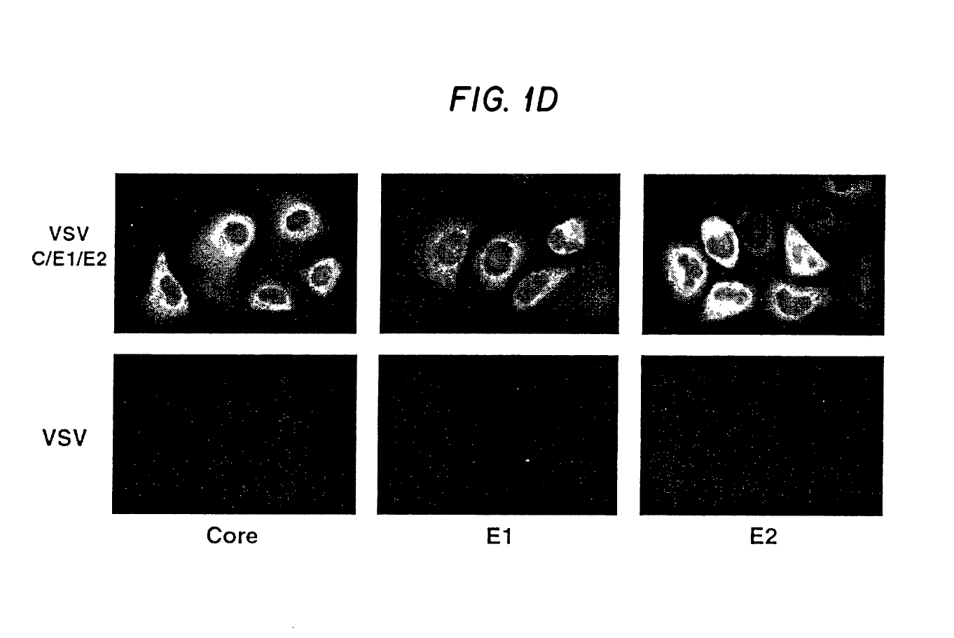

To evaluate whether recombinant (r) VSV could be utilized for the potential development of HCV-related vaccines and immunotherapies, we cloned in the entire structural region containing Core, E1 and E2 of HCV 1b (amino acids 1-746; accession number D89815(1)) into a cDNA representing the VSV genome (pVSV-XN2). To obtain recombinant VSV, the resultant plasmid (pVSV-HCV/C/E1/E2) was transfected with VSV N, P and L genes into BHK cells and virus was subsequently recovered (FIG. 1A). The Core, E1, and E2 region (amino acid residues 1-746) of the HCV polypeptide (NIHJ1 provided by T. Miyamura) was cloned into the XhoI and NheI sites of the rVSV replicon vector pVSV-XN2 (provided by J. Rose, prepared as describe in Lawson et al. (11)). This region of HCV was amplified by PCR using the forward primer 5′-CTCGTAGCTCGAGCATCATGAGCACAAATC-3′(SEQ ID NO: 1) which contains an XhoI restriction site and the reverse primer 5′-ACCAAGTTCTCTAGACTAAGCCTCGGCCTGGGCTAT-3′ (SEQ ID NO:2) which contains an XbaI site. This PCR product was cloned into pcDNA3.1NT-GFP (Invitrogen,; Carlsbad, Calif.) according to the TA cloning protocol. The new vector, pGFPC,E1,E2, was digested with XbaI and partially digested with XhoI and then inserted into pVSV-XN2. Recovery of recombinant VSV and the construction of VSV-GFP have been previously described (18).

Viable recombinant VSV containing the coding region of the HCV structural proteins (referred to as VSV-HCV-C/E1/E2) was plaque purified and exhibited similar growth properties to recombinant VSV expressing green fluorescent protein (VSV-GFP) when examined by growth curve analysis. However, VSV-HCV-C/E1/E2 did demonstrate slight growth attenuation and delayed cytopathic effect (FIG. 1B). BHK cells were infected at an m.o.i. (multiplicity of infection) of 1 for 30 min in serum-free DMEM which was then replaced with DMEM (Cellgro; Suwanee, Ga.) and 10% Fetal Bovine Serum (Gibco-BRL; Gaithersburg, Md.). 100 μl of cell medium was collected at 6, 12, 18, and 24 h post-infection. 100 μl of uninfected medium was replaced after each timepoint. Virus titers were determined by plaque assay.

To determine whether the recovered rVSV expressed HCV proteins, BHK or human liver derived Huh-7 cells (obtained from the ATCC) were infected with VSV-GFP or VSV-HCV-C/E1/E2 at a multiplicity of infection (m.o.i.) of 1. Infected cells were lysed 18 hours later and extracts analyzed by SDS-PAGE prior to being transferred to nitrocellulose. To detect HCV proteins, membranes were incubated with anti-Core, E1 or E2 antibody, while analysis of VSV protein expression was performed using anti-VSV mouse antiserum. HCV proteins were detected by anti-Core monoclonal antibody (Biogenesis, Poole, United Kingdom), anti-E1 (previously described (19), a gift from S. Polyak), and anti-E2 (polyclonal from C. Rice (20)). FIG. 1C indicates that VSV-HCV-C/E1/E2, but not VSV-XN2 infected cell lysates, efficiently expressed the HCV structural proteins. Since each of the antibodies reacted to their corresponding HCV products of the expected size (core, 21 kDa; E1, 35 kDa; E2, 68 kDa) in cells infected with VSV-HCV-C/E1/E2, we conclude that the HCV encoded C/E1/E2 polypeptide is efficiently expressed and post-translationally cleaved authentically into the individual structural proteins. Confirmation of high level HCV gene expression was achieved using immunofluorescent analysis of Huh-7 cells infected with VSV-XN2 or VSV-HCV-C/E1/E2 (FIG. 1D). Localization of HCV structural proteins was determined by immunofluorescence using monoclonal antibodies specific for Core, E1 (21, a gift from H. Greenberg), and E2 (22, a gift from M. Kohara). Briefly, Huh-7 cells were infected with VSV-XN2 or VSV-C/E1/E2 at an m.o.i. of 10 for 5 hrs and then fixed in 1% paraformaldehyde. The cells were incubated in 1:50 dilutions of primary antibody in 0.1% Brij-97/PBS for 2h at 4° C., washed with PBS-200 mM glycine, and then incubated with FITC-conjugated goat anti-mouse (1:100; Gibco-BRL) in 0.1% Brij-97/PBS for 1 h at 4° C. Immunostained cells were washed three time in PBS and treated with Slowfade Anti-Fade kit (Molecular Probes; Eugene, Oreg.).

VSV proteins were detected by polyclonal VSV mouse antiserum (18).

Antibody raised to HCV core strongly reacted to the perinuclear region of the cell, as previously reported for the HCV structural proteins in mammalian cells. In addition, antibody to E1 and E2 also indicated that these putative HCV glycoproteins also resided predominantly in the cytoplasm and not on the surface of the cell. Previous studies have indicated that E1 and E2 form non-covalent heterodimers, which reside as prebudding complexes in the endoplasmic reticulum (ER). Collectively, these data indicate that VSV is an efficient vehicle for the expression of HCV proteins

### EXAMPLE 2

Generation and Characterization of HCV-Like Particles (HCV-LPs).

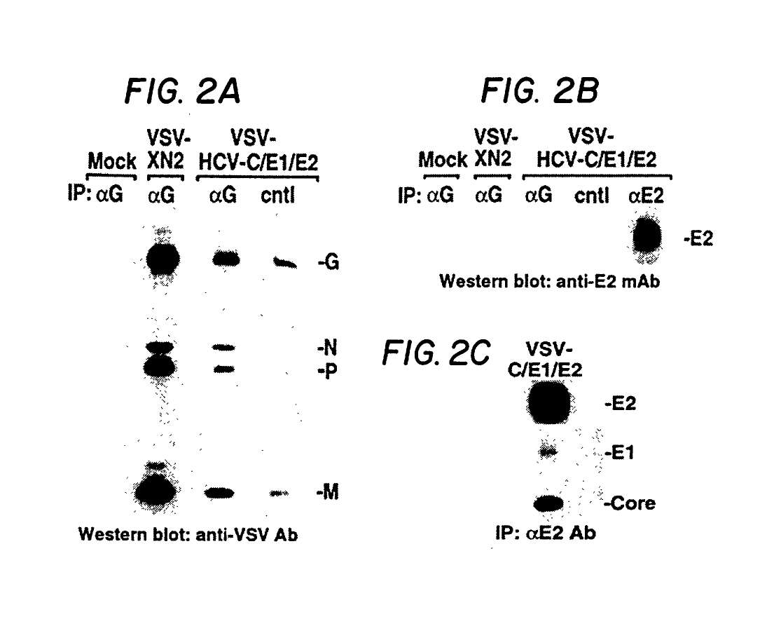

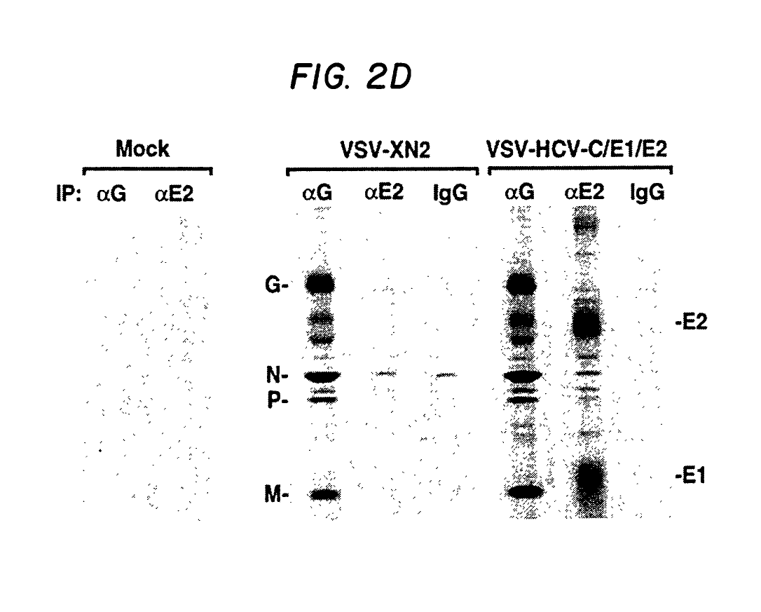

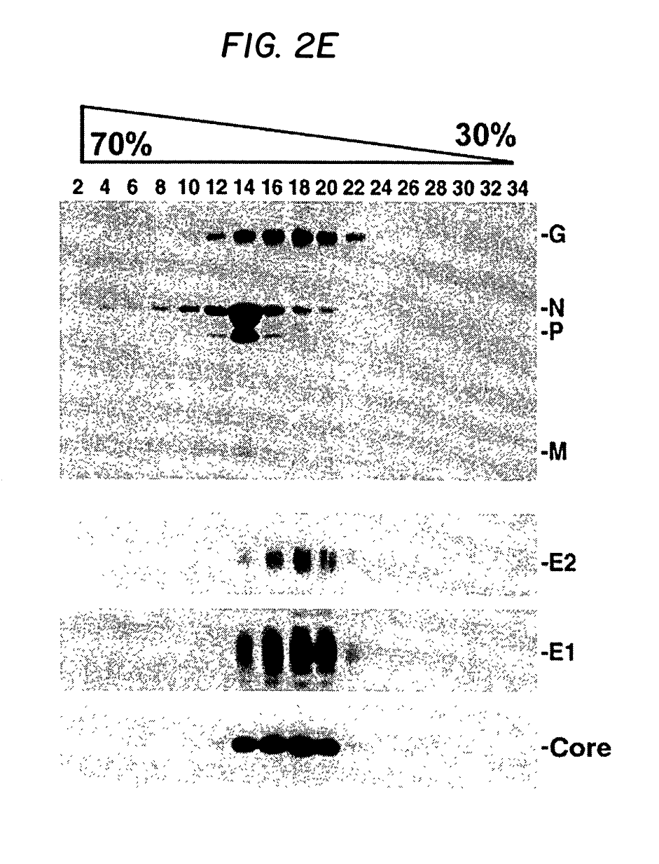

Evidence indicated that viable VSV was successfully generated to express HCV structural proteins Core, E1 and E2 to high levels. To evaluate the potential interactions of HCV C, E1 and E2 with one another as well as with VSV proteins, BHK cells were infected (m.o.i of 10) in the presence of ³⁵S-methionine/cysteine. After 18 hours, cells were lysed and protein extracts subjected to immunoprecipitation using antibody to VSV proteins or HCV E2. FIG. 2A indicates that immunoprecipitation of VSV-HCV-C/E1/E2 infected cell extracts with antibody to E2. VSV structural proteins N, P and M could be co-immunoprecipitated using the anti-G antibody. However, re-probing the blot with mouse anti-E2 antibody did not reveal detectable E2 protein in medium precipitated with anti-G, indicating that the released E1/E2 probably did not constitute a physical component of the VSV-HCV-C/E1/E2 virus (FIG. 2B). Precipitation of tissue cultured medium from VSV-HCV-C/E1/E2 infected cells with goat anti-E2 antibody, followed by immunoblotting with a mouse antibody raised to E2 confirmed the presence of the HCV envelope protein in the medium (FIG. 2B). Thus, although the HCV structural proteins are readily detectable in the medium, these proteins do not appear to be strongly associated with VSV-HCV-C/E1/E2 or to form chimeric viruses. This is most likely due to the HCV envelope products predominantly residing in the ER of the cell in addition to lacking C-terminal regions of VSV G critically required for incorporation into VSV particles as they dissociate from the cell membrane.

As indicated by the earlier immunofluorescence and immunoblot studies, HCV structural proteins were predominantly found in the cell lysate fraction rather than the medium (FIGS. 1C and 1D). To explore the association of intracellular HCV and VSV proteins, BHK cells were infected at an m.o.i. of 10 with VSV-XN2 or VSV-HCV-C/E1/E2. Four hours post-infection, cells were labeled with 35S-methionine/cysteine for another 12 hours before being lysed. Cell extracts precipitated with a mouse anti-E2 mAb confirmed strong association of HCV E1 with E2, but not with any VSV proteins (FIG. 2D). Reciprocal co-immunoprecipitation studies using mouse antiserum to VSV also indicated little or no association of HCV E1 and E2 with VSV products, again indicating that HCV proteins are not strongly coupled with VSV complexes (FIG. 2D).

To confirm the interaction of the HCV structural proteins as a possible virion and to determine if they could be purified from VSV protein complexes, cell lysates from VSV and VSV-C/E1/E2 infected BHK cells were clarified by centrifugation and layered onto a continuous 30-70% sucrose equilibrium gradient. Fractions were collected from the bottom and analyzed by Western blot for expression of Core, E1, E2, and VSV proteins (FIG. 2E).

BHK cells were infected at an m.o.i. of 0.1 for 18 h and then lysed in 50 mM Tris-HCl, pH 7.5, 50 mM NaCl, 0.1% NP-40, 1 mM PMSF, 10 ug/ml aprotinin, 10 μg/ml leupeptin, and 0.5 mM EDTA. The lysates were clarified and then spun through 30% sucrose in 50 mM Tris, 100 mM NaCl for 6 h at 150,000× g at 4° C. The pellets were resuspended and layered onto a continuous 30-70% sucrose gradient and spun for 22h at 150,000× g. One ml fractions were collected from the bottom, diluted in 50 mM Tris and 100 mM NaCl and then centrifuged for 2.5 h at 150,000× g. Fraction pellets were resuspended and analyzed by SDS-PAGE and immunoblot analysis.

The HCV proteins Core, E1, and E2 all predominantly sedimented in fractions 16-20, whereas the VSV proteins N, P, and M localized to fraction 14. This would indicate that Core, E1, and E2 form a complex that can be isolated from cell lysates and partially purified from VSV protein complexes. VSV's G protein does not exclusively sediment with other VSV proteins, instead, it appears to also be found in fractions containing the HCV structural proteins. This may indicate that some G protein may be taken up into HCV-like particles when they form in the endoplasmic reticulum. Although the VSV G protein is synthesized and processed through the ER, it is predominantly localized to the cell surface where VSV buds. This lack of strong co-localization and minimal co-immunoprecipitation would suggest that if VSV G is present in HCV-LPs, it is at very low levels. No HCV proteins were detected in control VSV gradients.

In order to further investigate formation of HCV-LPs, goat anti HCV E2 antibody was used to immunoprecipitate gradient purified HCV-LPs (FIG. 2C). After several washes, complexes were resolved using polyacrylamide gels and transferred to membranes for immunoblotting using anti-HCV Core and E1 mouse antibodies. FIG. 2C reveals that HCV Core and E1 could be detected in coimmunoprecipitation experiments of gradient purified HCV-LPs using anti-E2 antibody, strongly indicating co-association of Core and E1 with E2.

Given the supportive biochemical data for the formation of HCV-LPs, the demonstration of physical evidence of particle formation was undertaken. Uninfected BHK cells and BHK cells infected with either VSV-XN2 or VSV-C/E1/E2 were analyzed by transmission electron microscopy (TEM) for the identification of hepatitis C virus-like particles. Previous studies have measured hepatitis C virions to be 50-80 nm in diameter when isolated from patient serum.

BHK cells were infected at an m.o.i. of 0.01 with VSV-XN2, VSV-C/E1/E2 or left uninfected. Cells were washed twice in PBS and then fixed in 2% paraformaldehyde/2.5% gluteraldehyde, washed twice in PBS, and then incubated in 1% osmium tetroxide for 1h. Cells were rinsed twice in PBS and then dehydrated in a series of ethanol dilutions (35, 50, 70, 95, and 100% EtOH). In a 1:1 ratio of EtOH to Spurr's resin, the cells were infiltrated for 24h, then embedded in 100% Spurr's resin for 1h and incubated at 60° C. for 24 h. Thin sections were stained in aqueous 4% uranyl acetate for 20 min followed by lead citrate.

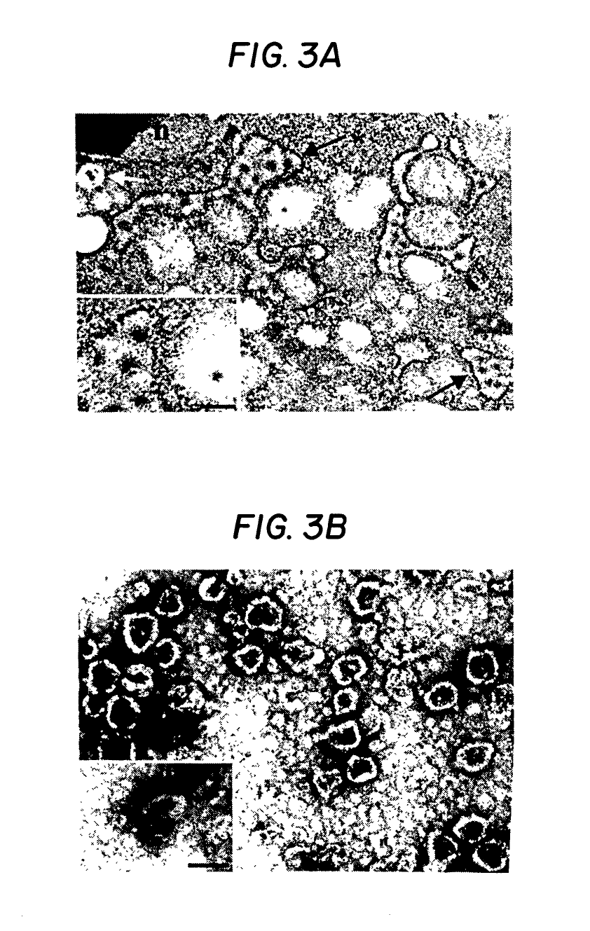

HCV-LPs generated in a baculovirus insect cell system measure to a similar size but are also noted to be polymorphic (23). When cells infected with VSV-C/E1/E2, but not VSV-XN2, were examined by TEM, HCV-LPs were found in cytoplasmic vacuoles of infected cells. Most of the particles were found in vacuoles generated from rough ER, but not exclusively. (FIG. 3A). HCV-LPs were differentiated from immature VSV complexes by both size and staining. HCV-LPs measure slightly larger than VSV and morphologically have dense cores with an envelope, whereas immature VSV appeared as a dense outer ring with a transparent core.

To directly correspond biochemical with biophysical data, HCV-LP dominant fractions from VSV-C/E1/E2 equilibrium sedimentation gradients and the respective VSV gradients, were examined by electron microscopy.

Purified HCV-LP particles were adsorbed to carbon coated copper grids and then negative stained with 2% uranyl acetate for 2-3min. For immunogold labeling in the inset, HCV-LPs were incubated with 1 μl of anti-E1 mAb for 30 min and then adsorbed to grids. Grids were washed 5 times in 1% BSA/PBS and then incubated with goat anti-mouse antibody conjugated to 15nm gold. Each grid was then washed 7 times in PBS and then stained with 2% uranyl acetate for 3-4 min.

Fractions from the VSV-C/E1/E2 gradients but not the VSV gradient contained virus-like particles. These particles were then labeled with anti-E1 mAb, which was subsequently bound by secondary antibody conjugated to 15nm gold particles. Immunogold labeling indicates that the virus-like particles isolated from fractions containing HCV Core, E1, and E2 proteins, do contain HCV E1 on the surface (FIG. 3B). Collectively, these data show that VSV expressing the HCV structural proteins can generate HCV-like particles and that these particles can be purified from cell extracts.

### EXAMPLE 3

VSV Expressing Core, E1, and E2 Can Generate an Immune Response to HCV.

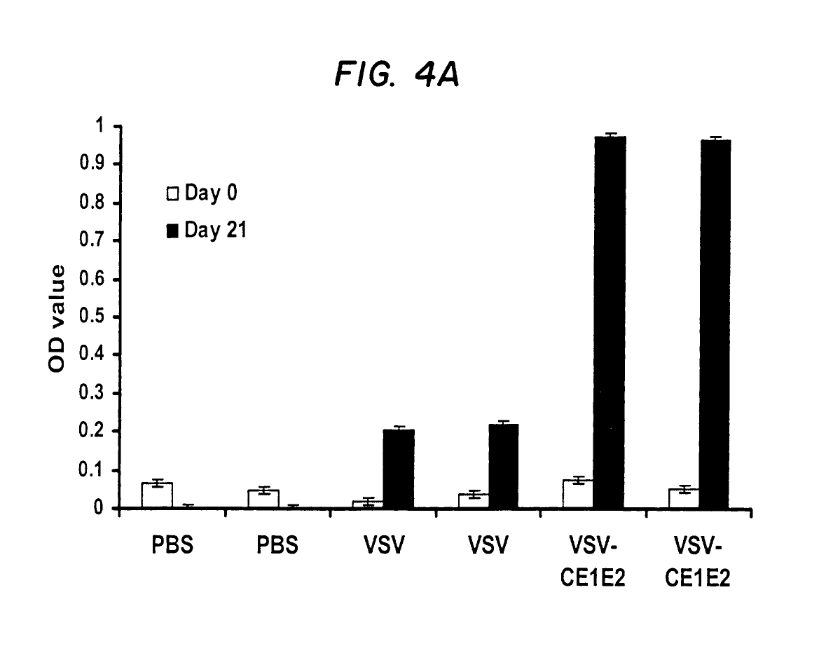

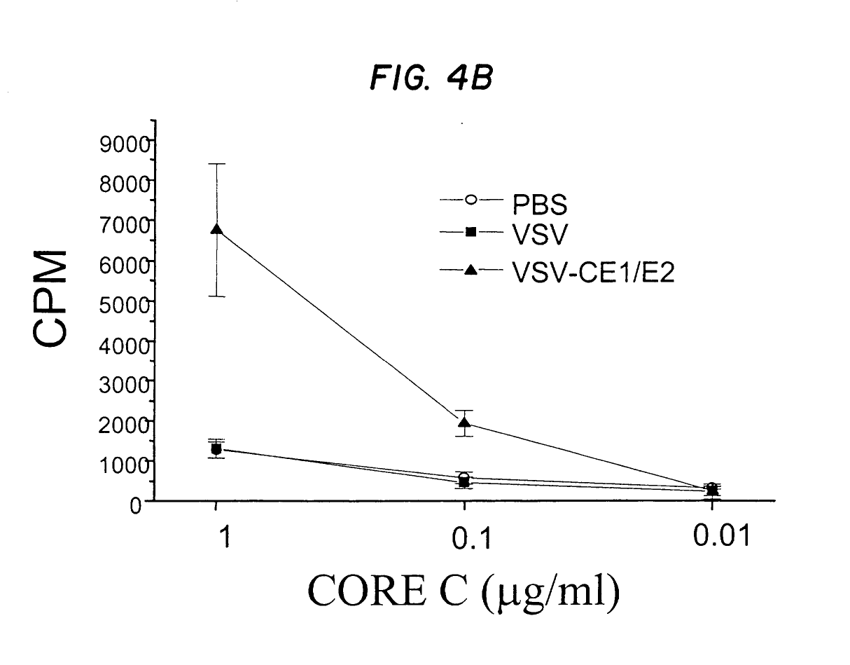

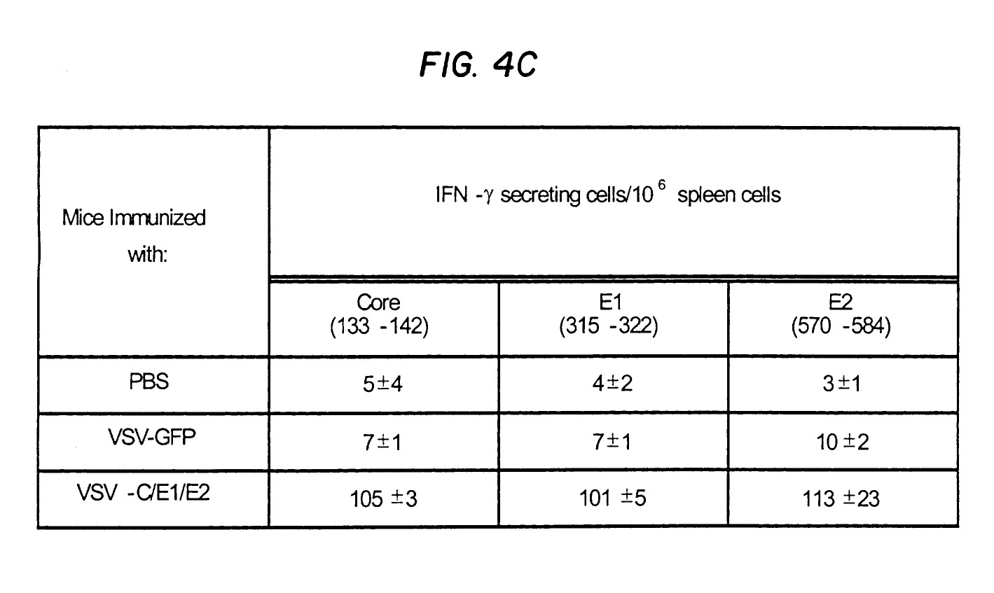

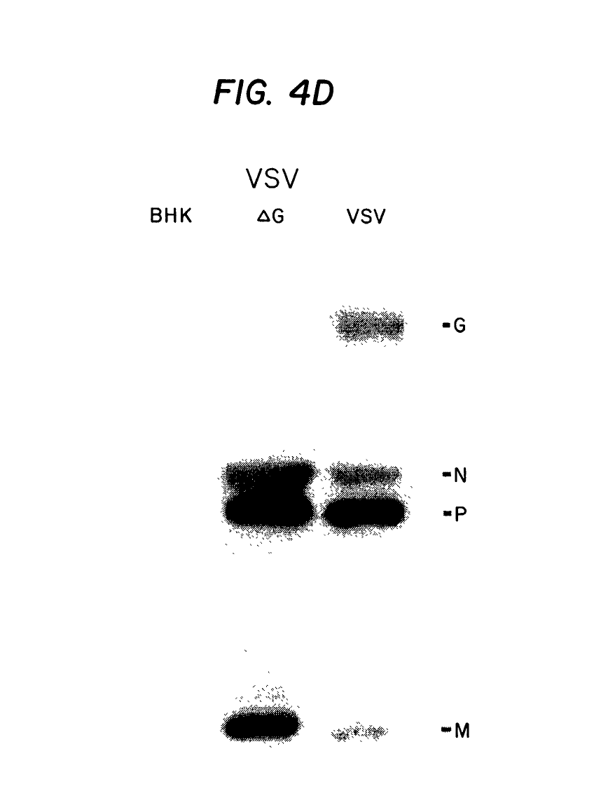

The immunologic impact of VSV infection has been extensively studied and shown to mediate a very strong humoral response and a long term cellular response. We examined whether VSV-C/E1/E2 could induce an immune response to the HCV structural proteins in mice. Balb/c mice were i.v. injected with 2.5×10⁶ pfu of VSV-GFP or VSV-C/E1/E2 or PBS, followed by 5×10⁶ pfu i.v. two weeks after the first injection. At 21 days post-initial injection, serum was collected from six mice in each group in order to conduct analysis for antibody production. The generation of antibodies to HCV Core and E2 was detected by enzyme-linked immunosorbent assay. 96 well plates were coated with purified Core and E2 proteins overnight and then blocked with 10% heat-inactivated FBS/PBS. After several washes with PBS/0.05% Tween, each plate was incubated with several dilutions of PBS, VSV-GFP, or VSV-C/E1/E2 vaccinated mouse serum for 2 hrs, followed by a 1:5000 dilution of goat anti-mouse secondary antibody conjugated to horseradish peroxidase. The ELISA plates were then developed with TMB substrate (Pharmingen) and read at 450 and 570 nm. All experiments were done in duplicate. These results indicate that mice vaccinated with VSV-C/E1/E2 generated antibody to the HCV Core protein and a robust humoral response to the E2 glycoprotein (FIGS. 4A and B). To further confirm VSV-C/E1/E2's ability to generate a CD4 response to HCV proteins, splenocytes from PBS, VSV-GFP, or VSV-C/E1/E2 vaccinated mice were harvested and pulsed with purified Core protein in a lymphoproliferative assay. The purified Core protein should only be presented by major histocompatibility complex class II (MHC class II) and therefore only activate CD4⁺ T cells causing their proliferation. As shown in FIG. 4C, purified Core protein induced the proliferation of CD4⁺ T cells from VSV-C/E1/E2 vaccinated mice but not VSV-GFP or PBS injected mice.

The generation of a multispecific cytotoxic T cell (CTL) response has previously been shown to be important for the clearance of HCV during acute infections in humans and chimpanzees. Therefore, in order to determine if VSV-C/E1/E2 generated a CD8⁺ T cell response, IFN-γ ELISPOT assays were performed on splenocytes isolated from PBS, VSV-GFP, or VSV-C/E1/E2 vaccinated mice two weeks after the second boost. Splenocytes from vaccinated mice were pulsed with 10 μg/ml of Core, E1, or E2 peptides that have been previously shown to activate cytotoxic T cells by chromium release assay. Only CTLs from VSV-C/E1/E2 vaccinated mice were activated by the HCV specific peptides as demonstrated by the production of IFN-γ (FIG. 4D).

### CONCLUSIONS

We have described herein the generation of recombinant VSV that expresses the HCV genotype 1b structural proteins Core, E1 and E2. Prior to the present invention, it was not known whether such VSV would express any of the HCV proteins, as the resultant recombinant virus could have been a lethal mutation and not grown. Furthermore, even if the virus were able to be generated, it was not clear that it would not be lethal to the host (mice). In addition, it was not clear that HCV VLPs would be produced or that VSV-HCV would be immunogenic. The recombinant viruses grew comparably to recombinant wild-type VSV and expressed high levels of fully processed recombinant HCV proteins. Our evidence indicates that the post-translational modifications experienced by the HCV proteins were likely authentic, having been produced in mammalian cells. Accordingly, the host environment facilitated assembly of the HCV proteins into virus-like particles, as determined both biophysically and biochemically. The ability to provoke cellular and humoral immune effect in an insect cell system (23) was dependent on the structural integrity of the particle formation. The overall conformation of VLPs presumably enhances their uptake by professional antigen presenting cells which are then processed by the MHC class I pathway to stimulate CTLs. Both humoral and T-cell responses were elicited by the VLPs against multiple viral proteins simultaneously, indicating that a strong, broad range immune response against multiple targets may be feasible. In these regards, VLPs have certain advantages over sub-unit based vaccines where adjuvants are required and CTL activity is low. The stimulation of CTL activity has been observed using recombinant human immunodeficiency virus (HIV) and human papilloma virus (HPV) like-particles in animal studies.

In addition to being an efficient vector for the expression of HCV-LPs in mammalian cells, VSV-HCV-C/E1/E2 exhibits characteristics indicating promise as a vaccine or immunogenic composition. Results demonstrated that VSV-HCV-C/E1/E2 could efficiently generate humoral and CTL activity to the Core, E1 and E2 proteins of HCV. We expect VSV-HCV as well as HCV-LPs to have efficacy in anti-HCV vaccine and immunotherapeutic strategies.

In describing preferred embodiments of the present invention, specific terminology is employed for the sake of clarity. However, the invention is not intended to be limited to the specific terminology so selected. It is to be understood that each specific element includes all technical equivalents, which operate in a similar manner to accomplish a similar purpose. Each published reference and patent cited herein is incorporated by reference as if each were individually incorporated by reference.

### REFERENCES

1. Aizaki, H., Y. Aoki, T. Harada, K. Ishii, T. Suzuki, S. Nagamori, G. Toda, Y. Matsuura, and T. Miyamura. 1998. Full-length complementary DNA of hepatitis C virus genome from an infectious blood sample. Hepatology 27:621-7.

2. Arichi, T., T. Saito, M. E. Major, I. M. Belyakov, M. Shirai, V. H. Engelhard, S. M. Feinstone, and J. A. Berzofsky. 2000. Prophylactic DNA vaccine for hepatitis C virus (HCV) infection: HCV-specific cytotoxic T lymphocyte induction and protection from HCV-recombinant vaccinia infection in an HLA-A2.1 transgenic mouse model. Proc Natl Acad Sci USA 97:297-302.

3. Brinster, C., S. Muguet, Y. C. Lone, D. Boucreux, N. Renard, A. Fournillier, F. Lemonnier, and G. Inchauspe. 2001. Different hepatitis C virus nonstructural protein 3 (Ns3)-DNA-expressing vaccines induce in HLA-A2.1 transgenic mice stable cytotoxic T lymphocytes that target one major epitope. Hepatology 34:1206-17. t&artType=abs&id=ajhep0341206&target=.

4. Cooper, S., A. L. Erickson, E. J. Adams, J. Kansopon, A. J. Weiner, D. Y. Chien, M. Houghton, P. Parham, and C. M. Walker. 1999. Analysis of a successful immune response against hepatitis C virus. Immunity 10:439-49.

5. Erickson, A. L., Y. Kimura, S. Igarashi, J. Eichelberger, M. Houghton, J. Sidney, D. McKinney, A. Sette, A. L. Hughes, and C. M. Walker. 2001. The outcome of hepatitis C virus infection is predicted by escape mutations in epitopes targeted by cytotoxic T lymphocytes. Immunity 15:883-95.

6. Farci, P., H. J. Alter, S. Govindarajan, D. C. Wong, R. Engle, R. R. Lesniewski, I. K. Mushahwar, S. M. Desai, R. H. Miller, N. Ogata, and et al. 1992. Lack of protective immunity against reinfection with hepatitis C virus. Science 258:135-40.

7. Forns, X., P. J. Payette, X. Ma, W. Satterfield, G. Eder, I. K. Mushahwar, S. Govindarajan, H. L. Davis, S. U. Emerson, R. H. Purcell, and J. Bukh. 2000. Vaccination of chimpanzees with plasmid DNA encoding the hepatitis C virus (HCV) envelope E2 protein modified the infection after challenge with homologous monoclonal HCV. Hepatology 32:618-25.

8. Heile, J. M., Y. L. Fong, D. Rosa, K. Berger, G. Saletti, S. Campagnoli, G. Bensi, S. Capo, S. Coates, K. Crawford, C. Dong, M. Wininger, G. Baker, L. Cousens, D. Chien, P. Ng, P. Archangel, G. Grandi, M. Houghton, and S. Abrignani. 2000. Evaluation of hepatitis C virus glycoprotein E2 for vaccine design: an endoplasmic reticulum-retained recombinant protein is superior to secreted recombinant protein and DNA-based vaccine candidates. J Virol 74:6885-92.

9. Houghton, M. 1996. Hepatitis C viruses, p. 1035-1058, Fields Virology, 3rd ed.

10. Kretzschmar, E., L. Buonocore, M. J. Schnell, and J. K. Rose. 1997. High-efficiency incorporation of functional influenza virus glycoproteins into recombinant vesicular stomatitis viruses. J Virol 71:5982-9.

11. Lawson, N. D., E. A. Stillman, M. A. Whitt, and J. K. Rose. 1995. Recombinant vesicular stomatitis viruses from DNA. Proc Natl Acad Sci USA 92:4477-81.

12. Pancholi, P., Q. Liu, N. Tricoche, P. Zhang, M. E. Perkus, and A. M. Prince. 2000. DNA prime-canarypox boost with polycistronic hepatitis C virus (HCV) genes generates potent immune responses to HCV structural and nonstructural proteins. J Infect Dis 182:18-27.

13. Roberts, A., E. Kretzschmar, A. S. Perkins, J. Forman, R. Price, L. Buonocore, Y. Kawaoka, and J. K. Rose. 1998. Vaccination with a recombinant vesicular stomatitis virus expressing an influenza virus hemagglutinin provides complete protection from influenza virus challenge. J Virol 72:4704-11.

14. Rose, N. F., P. A. Marx, A. Luckay, D. F. Nixon, W. J. Moretto, S. M. Donahoe, D. Montefiori, A. Roberts, L. Buonocore, and J. K. Rose. 2001. An effective AIDS vaccine based on live attenuated vesicular stomatitis virus recombinants. Cell 106:539-49.

15. Schlereth, B., J. K. Rose, L. Buonocore, V. ter Meulen, and S. Niewiesk. 2000. Successful vaccine-induced seroconversion by single-dose immunization in the presence of measles virus-specific maternal antibodies. J Virol 74:4652-7.

16. Schnell, M. J., L. Buonocore, E. Kretzschmar, E. Johnson, and J. K. Rose. 1996. Foreign glycoproteins expressed from recombinant vesicular stomatitis viruses are incorporated efficiently into virus particles. Proc Natl Acad Sci USA 93:11359-65.

17. Seong, Y. R., S. Choi, J. S. Lim, C. H. Lee, C. K. Lee, and D. S. Im. 2001. Immunogenicity of the E1E2 proteins of hepatitis C virus expressed by recombinant adenoviruses. Vaccine 19:2955-64.

18. Fernandez, M., M. Porosnicu, D. Markovic, and G. N. Barber. 2002. Genetically engineered vesicular stomatitis virus in gene therapy; appplication for treatment of malignant disease. J. Virol. 76:895-904.

19. Polyyak, S. J., K. S. Khabar, D. M. Paschal, H. J. Ezelle, G. Duverlie, G. N. Barber, D. E. Levy, N. Mukaida, D. R. Gretch. 2001. Hepatitis C virus nonstructural 5A protein induces interleukin-8, leadint to partial inhibition of the interferon-induced antiviral response. J. Virol. 75:6095-106.

20. Lin, C., B. D. Lindenbach, B. M. Pragai, D. W. McCourt, and C. M. Rice. 1994 Processing in the hepatitis C virus E2-NS2 region: identification of p7 and two distinct E2 specific products with different C termini. J. Virol 68:5063-73.

21. Hsu, H. H., M. Gonzalex, S. K. Foung, S. M. Feinstone, H. B. Greenberg. 1991. Antibodies to hepatitis C virus in low-risk blood donors: implications for counseling positive donors. Gastroenterology 101: 1724-7.

22. Kaito, M., S. Watanabe, K Tsukiyama-Kohara, K. Yamaguchi, Y. Kobayashi, M. Konishi, M. Yokaoi, S. Ishida, S. Suzuki, and M. Kohara. 1994. Hepatitis C virus particle detected by immunoelectron microscopic study. J. Gen Virol. 75 (Pt 7):1755-60.

23. Baumert, T. F., S. Ito, D. T. Wong, T. J. Liang. 1998. Hepatitis C virus structural proteins assemble into viruslike particles in insect cells. J. Virol. 72:3827-36.

What is claimed is:

1. A recombinant vesicular stomatitis virus (VSV) that expresses one or more HCV structural proteins selected from the group consisting of HCV Core, E1 protein and E2 protein.

2. The recombinant VSV of claim 1 that expresses HCV Core, E1 protein and E2 polyprotein.

3. The VSV of claim 2 that is designated VSV-HCV-C/E1/E2.

4. The expression product of the VSV of claim 1.

5. The expression product of claim 4 that is HCV virus-like particles (HCV-LPs).

6. A recombinant VSV having incorporated into its genome the contiguous Core, E1 and E2 coding region of HCV.

7. A recombinant VSV that produces virus-like particles with properties of HCV virions (HCV-LPs).

8. The recombinant VSV of claim 7 wherein the virus-like particles have the ability to elicit a cell-mediated and/or humoral immune response to HCV when administered to a mammal.

9. The recombinant VSV of claim 8 that elicits cell-mediated immune responses to HCV Core, E1 and E2 proteins.

10. The recombinant VSV of claim 8 that elicits humoral immune responses to HCV Core and E2 proteins.

11. A vaccine or immunogenic composition comprising the recombinant VSV of claim 2.

12. The vaccine or immunogenic composition of claim 11 comprising the recombinant VSV that is designated VSV-HCV-C/E1/E2.

13. A vaccine or immunogenic composition comprising HCV virus-like particles (HCV-LPs) produced by the recombinant VSV of claim 1.

14. The vaccine or immunogenic composition of claim 13 wherein the recombinant VSV is VSV-HCV-C/E1/E2.

15. A method of inducing an immune response to HCV in an individual, said method comprising administering to the individual an effective amount of the vaccine or immunogenic composition of claim 11.

16. A method of inducing an immune response to HCV in an individual, said method comprising administering to the individual an effective amount of the vaccine or immunogenic composition of claim 12.

17. A method of inducing an immune response to HCV in an individual, said method comprising administering to the individual an effective amount of the vaccine or immunogenic composition of claim 13.

18. A method of inducing an immune response to HCV in an individual, said method comprising administering to the individual an effective amount of the vaccine or immunogenic composition of claim 14.

19. A method for prophylaxis or treatment of HCV infection, comprising administering to an individual in need of prophylaxis or treatment an effective amount of the composition of claim 12.

20. A method for prophylaxis or treatment of HCV infection, comprising administering to an individual in need of prophylaxis or treatment an effective amount of the composition of claim 14.

21. Isolated mammalian cell(s) infected with the recombinant VSV of claim 1.


SEQUENCE LISTING (2 sequences)

SEQ ID NO: 1 - DNA, 30 bp, Artificial Sequence
  oligonucleotide primer
ctcgtagctcgagcatcatgagcacaaatc

SEQ ID NO: 2 - DNA, 36 bp, Artificial Sequence
  oligonucleotide primer
accaagttctctagactaagcctcggcctgggctat